(19) United States
(12) Patent Application Publication
(10) Pub. No.: US 2005/0287185 A1
(43) Pub. Date: Dec. 29, 2005

(54) Extended release oxybutynin formulation

(76) Inventors: David Wong, Milpitas, CA (US); Xiu Xiu Cheng, Weston, FL (US); Chih-Ming Chen, Taipei (TW); Steve Jan, Coral Springs, FL (US); Aaron Dely, Lighthouse Point, FL (US); Dacheng Tian, Miramar, FL (US)

Correspondence Address: HEDMAN & COSTIGAN P.C., 1185 AVENUE OF THE AMERICAS, NEW YORK, NY, 10036, US

(21) Appl. No.: 10/902,739

(22) Filed: Jul. 29, 2004

Related U.S. Application Data
(60) Provisional application No. 60/582,274, filed on Jun. 23, 2004.

(51) Int. Cl. A61K 9/48

(52) U.S. Cl. 424/423

(57) ABSTRACT

A controlled release oxybutynin tablet that employs a homogeneous core with less than 50% of a water swellable polymer and a semi-permeable membrane that surrounds the homogeneous core.

### CROSS REFERENCE TO RELATED APPLICATIONS

This application claims priority to U.S. Provisional Patent Application Ser. No. 60/582274, filed Jun. 23, 2004.

### BACKGROUND OF THE INVENTION

The present invention relates to oral controlled release dosage formulations containing the drug oxybutynin. More specifically, the present invention relates to an oral osmotic dosage form comprising a homogeneous or unitary core that contains the drug oxybutynin or a pharmaceutically acceptable salt, as described in the United States Pharmacopoeia, National Formulary, Oxybutynin Chloride, (1995) pp. 1127-1128, which is incorporated herein by reference. Oxybutynin Chloride is known chemically as 4-diethylamino-2-butynyl-phenylcyclohexylglycolate hydrochloride. Oxybutynin is currently marketed as DITROPAN® and DITROPAN® XL tablets by ALZA Corp. Each DITROPAN® tablet contains 5 mg of oxybutynin. The usual dose in the pharmacological management is repeated doses from two-to-four times a day for oxybutynin. DITROPAN® XL tablets contain either 5, 10 or 15 mg unit doses of oxybutynin and is dosed once a day. Oxybutynin chloride is describe in the 54th Edition of the Physicians' Desk Reference, copyright 2000, p. 507 as a white crystalline solid which is readily soluble in both water and acids, but relatively insoluble in alkalis. The present invention provides for the controlled release of therapeutic amounts of oxybutynin over a twelve to twenty-four time period thereby allowing for twice a day or once a day dosing.

Urinary incontinence is a common problem among many elderly individuals and women. A well known treatment for incontinence consists of the administration of a smooth muscle relaxant such as oxybutynin to a patient in need of such treatment. In the past such treatment has consisted of two to four administrations of the drug throughout the day.

Many techniques have been used to provide controlled and extended-release pharmaceutical dosage forms in order to maintain therapeutic serum levels of medicaments and to minimize the effects of missed doses of drugs caused by a lack of patient compliance.

The prior art teaches extended release tablets which have an osmotically active drug core surrounded by a semipermeable membrane. These tablets function by allowing a fluid such as gastric or intestinal fluid to permeate the coating membrane and dissolve the active ingredient so it can be released through a passageway in the coating membrane or if the active ingredient is insoluble in the permeating fluid, pushed through the passageway by an expanding agent such as a hydrogel. Some representative examples of these osmotic tablet systems can be found in U.S. Pat. Nos. 3,845,770; 3,916,899; 4,034,758; 4,077,407 and 4,783,337. U.S. Pat. No. 3,952,741 teaches an osmotic device wherein the active agent is released from a core surrounded by a semipermeable membrane only after sufficient pressure has developed within the membrane to burst or rupture the membrane at a weak portion of the membrane. The basic osmotic device described in the above cited patents have been refined over time in an effort to provide greater control of the release of the active ingredient. For example U.S. Pat. Nos. 4,777,049 and 4,851,229 describe an osmotic dosage form comprising a semipermeable wall surrounding a core. The core contains an active ingredient and a modulating agent wherein the modulating agent causes the active ingredient to be released through a passageway in the semipermeable membrane in a pulsed manner. Further refinements have included modifications to the semipermeable membrane surrounding the active core such as varying the proportions of the components that form the membrane, e.g., U.S. Pat. Nos. 5,178,867; 4,587,117 and 4,522,625 or increasing the number of coatings surrounding the active core, e.g., U.S. Pat. Nos. 5,650,170 and 4,892,739.

The prior art discloses dosage formulations that can be used to deliver the drug oxybutynin in a sustained manner for twelve to twenty four hours. For example Japanese Kokai Patent Application 6-9388, which is assigned to Kodama Kabushiki Kaisha and is incorporated herein by reference, discloses a dosage formulation for the oral administration of oxybutynin that can be administered to a patient once or twice a day. The dosage formulation consists of a blend of oxybutynin, an acidic substance such as an organic acid, a gel forming substance such as hydroxypropyl methylcellulose and a higher alcohol (having more than twelve carbons) such as lauryl or stearyl alcohol. Other examples of sustained release dosage formulations that contain oxybutynin can be found in U.S. Pat. Nos. 5,674,895; 5,840,754 and 5,912,268. These patents disclose oxybutynin dosage forms that have a large amount of a gelling polymer and/or a bilayered osmotic core.

Extended release oxybutynin formulations with improved dissolution properties are therefore a desirable addition to the medical treatment of urinary incontinence. For example, DITROPAN® XL (ALZA Corp., 5, 10 and 15 mg unit doses) is an extended release oxybutynin formulation available as osmotic based dosage forms. DITROPAN® XL is prepared as an osmotic pump formulation. Specifically, DITROPAN ® XL is prepared as an osmotically active drug core surrounded by a semipermeable membrane. The core itself is divided into two layers: an “active” layer containing the drug, and a “push” layer containing pharmacologically inert (but osmotically active) components. The membrane surrounding the tablet is permeable to water but not to the drug or osmotic excipients. As water from the gastrointestinal tract enters the osmotically active material, the tablet pressure increases in the osmotic layer and “pushes” against the drug layer, resulting in the release of drug through a small drilled orifice in the membrane on the drug side of the tablet.

Other osmotic pump devices and formulations for administering oxybutynin are described in U.S. Pat. Nos. 5,674,895; 5,912,268 and 5,840,754 (Guittard et al.).

In view of the above discussion, it will be appreciated by those skilled in the art to which this invention pertains, that a need exists for easily manufactured dosage forms that can deliver oxybutynin in a controlled release to a patient in clinical need of this drug, and methods of use therapy.

The above prior art disclosures of sustained release oxybutynin dosage formulations all require a large amount of a gelling polymer which results in a large dosage formulation that is often difficult for patients to swallow or they require a bilayer core that is difficult to manufacture.

It is an object of the present invention to provide a controlled or sustained release dosage form of oxybutynin which substantially overcomes the deficiencies and omissions associated with the prior art.

It is a further object of the present invention to provide a dosage form for orally administering oxybutynin in a controlled release dose for the non-surgical treatment of incontinence in a human afflicted with incontinence.

It is also an object of the present invention is to provide a pharmacologic composition comprising oxybutynin, its racemate, its R-enantiomer and/or its S-enantiomer enantiomer administrable to humans for lessening the incidence of incontinence.

It is a further object of the invention to provide a pharmacologic composition comprising oxybutynin indicated for the pharmacologic management of incontinence. Other objects, features and advantages of this invention will be more apparent to those of ordinary skill in the art from the following detailed specification, taken in conjunction with the drawings and the accompanying claims.

In accordance with the above-mentioned objects and others, the present invention in certain embodiments is directed to a controlled release solid oral dosage form comprising oxybutynin, or a pharmaceutically acceptable salt thereof, that is suitable for providing once-a-day administration of the drug, wherein the dosage form provides a mean time to maximum plasma concentration (Tₘₐₓ) of the drug at from about 4 hours to about 18 hours after administration. The dosage form comprises the drug, a pharmaceutically acceptable polymer and a membrane. In certain preferred embodiments the dosage form comprises a tablet.

It is an object of the present invention to overcome the problems associated with the prior art sustained release oxybutynin dosage formulation. More specifically, it is an object of the present invention to provide an oral oxybutynin dosage formulation that can be administered once or twice daily, that has a low amount of gelling polymer and that has a unitary or homogeneous core.

### SUMMARY OF THE INVENTION

In preferred embodiments, the controlled release solid oral dosage form of the present invention is a tablet comprising:
(a) a homogeneous core comprising:
   (i) an effective amount of oxybutynin or a pharmaceutically acceptable salt;
   (ii) less than 50%, preferably less than 30% and most preferably less than 15% by weight of the core of a water swellable or gelling material;
(b) optionally a seal coat surrounding the core;
(c) a semi-permeable membrane that surrounds the core; and
(d) at least one passageway in the semi-permeable membrane.

The daily dose of oxybutynin may vary, e.g., from about 0.1 mg to about 100 mg. Such daily dose may be contained in one controlled-release dosage form of the invention, or may be contained in more than one such dosage form. For example, a controlled-release oxybutynin dosage form may be formulated to contain about 5, 10 or 15 mg of the drug, and two dosage forms may be administered together to provide a desired once-a-day oxybutynin dose. The daily dose of the drug (e.g., oxybutynin or pharmaceutically acceptable salt thereof) may range from about 0.1 mg to about 100 mg, from about 1 mg to about 60 mg, or from about 5 mg to about 30 mg, depending on the clinical needs of the patient.

In preferred embodiments, the controlled release solid oral dosage form of the present invention provides a mean time to maximum plasma concentration of oxybutynin at from about 4 to about 18 hours after administration, preferably at from about 6 hours to about 14 hours after administration, and most preferably at from about 6 to about 12 hours after administration.

In certain other embodiments, the once-a-day oxybutynin therapy of the present invention may be used concomitantly with other anti-spasmodic and/or anti-cholinergic agents, and/or other agents for the treatment of incontinence or diseases related thereto.

The term “oxybutynin” as it is used herein means oxybutynin base, its racemate, its R-enantiomer, its S-enantiomer, oxybutynin chloride or any other pharmaceutically acceptable salt.

The term “dosage form” as it is used herein means at least one unit dosage form of the present invention (e.g. the daily dose of the oxybutynin can be contained in 2 unit dosage forms of the present invention for single once-a-day administration).

The term “sustained release” and “controlled release” are used interchangeably in this application and are defined for purposes of the present invention as the release of the drug from the dosage form at such a rate that when a once-a-day dose of the drug is administered in the sustained release or controlled-release form, blood (e.g., plasma) concentrations (levels) of the drug are maintained within the therapeutic range but below toxic levels over a period of time from about 12 to about 24 hours. When the drug used in the present invention is oxybutynin chloride, the controlled release solid oral dosage form containing such drug is also referred to as “Oxybutynin HCL E.R.”

The term “Cₘₐₓ” is the highest plasma concentration of the drug attained within the dosing max interval, i.e., about 24 hours.

The term “Tₘₐₓ” is the time period which elapses after administration of the dosage form at which the plasma concentration-of the drug attains the highest plasma concentration of drug attained within the dosing interval (i.e., about 24 hours).

The term “AUC₀₋ₜ” as used herein, means area under the plasma concentration-time curve, as calculated by the trapezoidal rule over time from 0 to t.

The term “single dose” means that the human patient has received a single dose of the drug formulation and the drug plasma concentration may have not achieved steady state.

The term “multiple dose” means that the human patient has received at least two doses of the drug formulation in accordance with the dosing interval for that formulation (e.g., on a once-a-day basis). Patients who have received multiple doses of the controlled release formulations of the invention may or may not have attained steady state drug plasma levels, as the term multiple dose is defined herein.

The term “mean”, when preceding a pharmacokinetic value (e.g. mean Tₘₐₓ) represents the arithmetic mean or geometric mean value of the pharmacokinetic value taken from a population of patients unless otherwise specified (e.g. geometric mean).

The term “inverse relationship” with respect to release rate and amount of polymer means that as the amount of polymer increases, the release rate decreases, and vice versa. When the membrane of the invention is disclosed as “surrounding” the core, it is understood that this does not preclude the existence of an intermediate layer between the membrane and the core, e.g. an intermediate film coating.

In certain preferred embodiments, the controlled release solid oral dosage form exhibits the following dissolution profiles when tested in USP type 2 apparatus at 50 rpm in 900 ml of simulated intestinal fluid (pH 6.5 potassium phosphate buffer) at 370° C.: from 0 to about 20% of oxybutynin or a pharmaceutically acceptable salt thereof released after 2 hours; from about 5% to about 40% of oxybutynin or a pharmaceutically acceptable salt thereof released after 4 hours; from about 20% to about 60% of oxybutynin or a pharmaceutically acceptable salt thereof released after 8 hours; from about 30% to about 80% of oxybutynin or a pharmaceutically acceptable salt thereof released after 12 hours; not less than about 40% of oxybutynin or a pharmaceutically acceptable salt thereof released after 16 hours; and not less than about 50% of oxybutynin or a pharmaceutically acceptable salt thereof released after 20 hours.

In certain preferred embodiments, the controlled release solid oral dosage form exhibits the following dissolution profiles when tested in USP type 2 apparatus at 50 rpm in 900 ml of simulated intestinal fluid (pH 7.5 potassium phosphate buffer and Tween 80) at 370° C.: from 0 to about 40% of oxybutynin or a pharmaceutically acceptable salt thereof released after 2 hours; from about 10% to about 50% of oxybutynin or a pharmaceutically acceptable salt thereof released after 4 hours; from about 30% to about 60% of oxybutynin or a pharmaceutically acceptable salt thereof released after 8 hours; from about 40% to about 80% of oxybutynin or a pharmaceutically acceptable salt thereof released after 12 hours; and not less than about 40% of oxybutynin or a pharmaceutically acceptable salt thereof released after 16 hours; and not less than about 50% of oxybutynin or a pharmaceutically acceptable salt thereof release after 20 hours.

In certain embodiments of the present invention, the controlled release oral dosage form of the oxybutynin or a pharmaceutically acceptable salt thereof provides a mean maximum plasma concentration (Cₘₐₓ) of the drug that is about 0.15 ng/ml to about 6 ng/ml, based on administration of a 15 mg once-a-day dose of oxybutynin, more preferably about 0.75 ng/ml to about 3.0 ng/ml, based on administration of a 15 mg once-a-day dose of oxybutynin.

In certain embodiments of the present invention, the controlled release oral dosage form of the oxybutynin or a pharmaceutically acceptable salt thereof provides a mean maximum plasma concentration (Cₘₐₓ) of the drug that is about 0.1 ng/ml to about 4 ng/ml, based on administration of a 10 mg once-a-day dose of oxybutynin, more preferably about 0.5 ng/ml to about 2.0 ng/ml, based on administration of a 10 mg once-a-day dose of oxybutynin.

In certain embodiments of the present invention, the controlled release oral dosage form of the oxybutynin or a pharmaceutically acceptable salt thereof provides a mean maximum plasma concentration (C_{final}) of the drug that is about 0.05 ng/ml to about 2 ng/ml, based on administration of a 5 mg once-a-day dose of oxybutynin, more preferably about 0.25 ng/ml to about 1.0 ng/ml, based on administration of a 5 mg once-a-day dose of oxybutynin.

In certain embodiments of the present invention, the controlled release dosage form of oxybutynin or a pharmaceutically acceptable salt thereof provides a mean AUC_{0-48 hr} that is about 7.5 ng.hr/ml to about 90 ng.hr/ml, based on administration of a 15 mg once-a-day dose of oxybutynin; and preferably about 15 ng.hr/ml to about 60 ng.hr/ml, based on administration of a 15 mg once-a-day dose of oxybutynin.

In certain embodiments of the present invention, the controlled release dosage form of oxybutynin or a pharmaceutically acceptable salt thereof provides a mean AUC_{0-48 hr} that is about 5 ng.hr/ml to about 60 ng.hr/ml, based on administration of a 10 mg once-a-day dose of oxybutynin; and preferably about 10 ng.hr/ml to about 40 ng.hr/ml, based on administration of a 10 mg once-a-day dose of oxybutynin.

In certain embodiments of the present invention, the controlled release dosage form of oxybutynin or a pharmaceutically acceptable salt thereof provides a mean AUC_{0-48 hr} that is about 2.5 ng.hr/ml to about 30 ng.hr/ml, based on administration of a 5 mg once-a-day dose of oxybutynin; and preferably about 5 ng.hr/ml to about 20 ng.hr/ml, based on administration of a 5 mg once-a-day dose of oxybutynin.

In certain preferred embodiments, the controlled release solid oral dosage form of the present invention, after oral administration of a single dose to a human patient, provides a mean plasma concentration of oxybutynin of from about 900 to about 2700 pg/ml at 4 hours after administration, from about 1500 to about 3000 pg/ml at 8 hours after administration; from about 1800 to about 3600 pg/ml at 12 hours after administration and from about 600 to about 2100 pg/ml at 24 hours after administration, based on a 15 mg dose of oxybutynin.

In certain preferred embodiments, the controlled release solid oral dosage form of the present invention, after oral administration of a single dose to a human patient, provides a mean plasma concentrations of oxybutynin of from about 1200 to about 2100 ng/ml at 4 hours after administration, from about 1800 to about 2700 ng/ml at 8 hours after administration; from about 2100 to about 3000 ng/ml at 12 hours after administration and from about 750 to about 1500 ng/ml at 24 hours after administration, based on a 15 mg/dose of oxybutynin.

In certain preferred embodiments, the controlled release solid oral dosage form of the present invention, after oral administration of a single dose to a human patient, provides a mean plasma concentration of oxybutynin of from about 600 to about 1800 pg/ml at 4 hours after administration, from about 1000 to about 2000 pg/ml at 8 hours after administration from about 1200 to about 2400 pg/ml at 12 hours after administration and from about 400 to about 1400 pg/ml at 24 hours after administration, based on a 10 mg dose of oxybutynin.

In certain preferred embodiments, the controlled release solid oral dosage form of the present invention, after oral administration of a single dose to a human patient, provides a mean plasma concentrations of oxybutynin of from about 800 to about 1400 ng/ml at 4 hours after administration, from about 1200 to about 1800 ng/ml at 8 hours after administration; from about 1400 to about 2000 ng/ml at 12 hours after administration and from about 500 to about 1000 ng/ml at 24 hours after administration, based on a 10 mg dose of oxybutynin.

In certain preferred embodiments, the controlled release solid oral dosage form of the present invention, after oral administration of a single dose to a human patient, provides a mean plasma concentration of oxybutynin of from about 300 to about 900 pg/ml at 4 hours after administration, from about 500 to about 1000 pg/ml at 8 hours after administration; from about 600 to about 2400 pg/ml at 12 hours after administration and from about 200 to about 700 pg/ml at 24 hours after administration, based on a 5 mg dose of oxybutynin.

In certain preferred embodiments, the controlled release solid oral dosage form of the present invention, after oral administration of a single dose to a human patient, provides a mean plasma concentrations of oxybutynin of from about 400 to about 700 ng/ml at 4 hours after administration, from about 600 to about 900 ng/ml at 8 hours after administration; from about 700 to about 1000 ng/ml at 12 hours after administration and from about 250 to about 500 ng/ml at 24 hours after administration, based on a 5 mg dose of oxybutynin.

In certain embodiments of the invention, the once-a-day administration of the oxybutynin dosage form provides a mean AUC_{0-24 hr} from about 80% to about 125%, preferably from about 90% to about 110% of the mean AUC_{0-24 hr} provided by once-a-day administration of an equivalent dose of a controlled release reference standard (DITROPAN® XL).

The dosage form of the present invention can provide therapeutic levels of oxybutynin for twelve to twenty-four hour periods. In a preferred embodiment, the dosage form can be administered once a day, and provides therapeutic levels of the drug throughout the day with peak plasma levels being obtained preferably from about 8 hours to about 14 hours after administration.

Other embodiments of the present invention may employ other conventional pharmaceutical excipients such as a diluent, lubricants, glidants and osmotic agents in the core and the semi-permeable membrane may include channeling agents, flux enhancers and plasticizers.

### BRIEF DESCRIPTION OF THE DRAWINGS

FIG. 1 is a graph which depicts the dissolution profile in simulated intestinal fluid (pH 6.5 potassium phosphate buffer and pH 7.5 phosphate buffer and 0.5% tween 80) of a 15 mg controlled release oxybutynin formulation as described in Example 2 and as tested according to the procedure described in United States Pharmacopoeia XXIII, Apparatus 2 @ 50 rpm.

FIG. 2 is a graph which depicts the dissolution profile in simulated intestinal fluid (pH 6.5 potassium phosphate buffer) of a 15 mg controlled release oxybutynin formulation as described in Examples 3 and 4 and as tested according to the procedure described in United States Pharmacopoeia XXIII, Apparatus 2 @ 50 rpm.

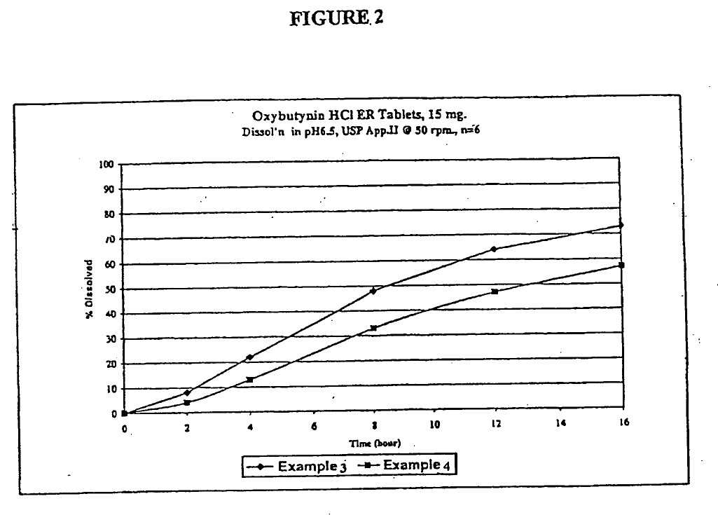

FIG. 3 is a graph which depicts the dissolution profile in simulated gastric fluid (SGF) of a 15 mg controlled release oxybutynin formulation as described in Example 3 and as tested according to the procedure described in United States Pharmacopoeia XXIII, Apparatus 2 @ 50 rpm.

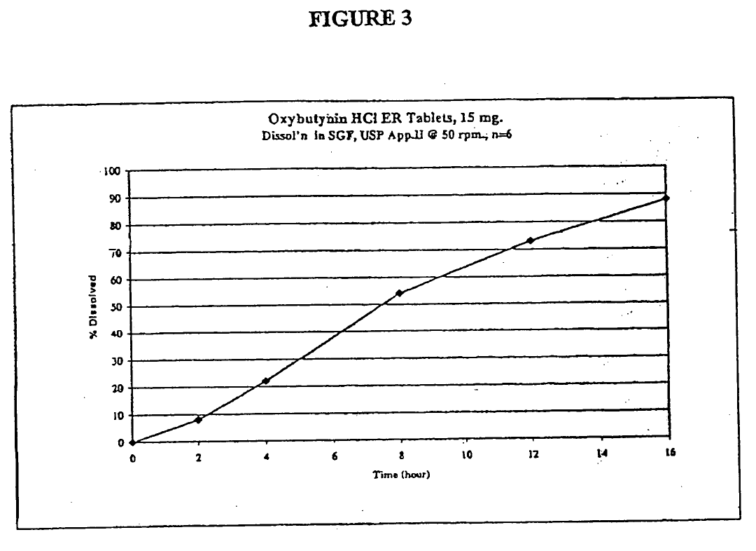

FIG. 4 is a graph which depicts the dissolution profile in simulated intestinal fluid (pH 7.5 phosphate buffer +0.5% tween 80) of a 15 mg controlled release oxybutynin formulation as described in Example 4 and as tested according to the procedure described in United States Pharmacopoeia XXIII, Apparatus 2 @ 50 rpm.

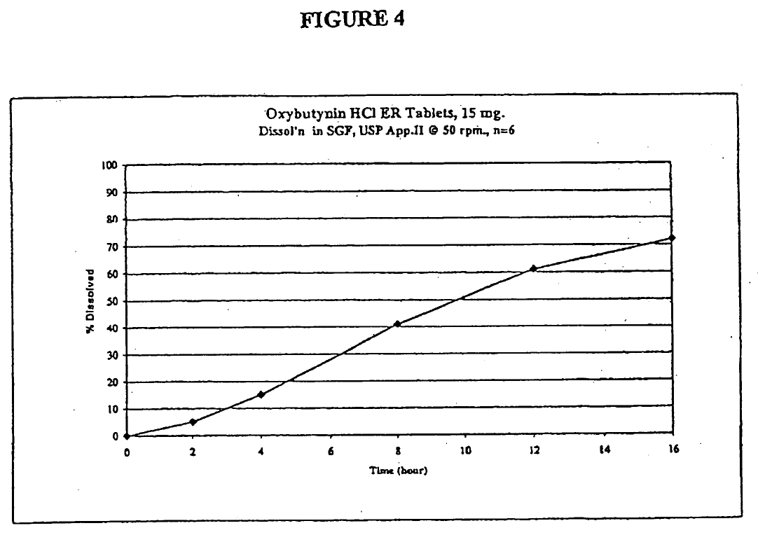

FIG. 5 is a graph which depicts the dissolution profile in simulated gastric fluid (SGF) of a 15 mg controlled release oxybutynin formulation as described in Example 4 and as tested according to the procedure described in United States Pharmacopoeia XXIII, Apparatus 2 @ 50 rpm.

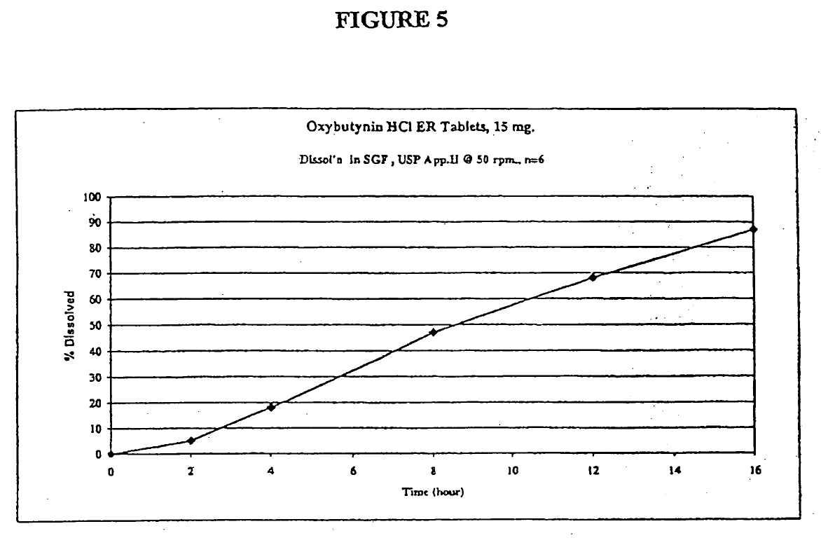

FIG. 6 is a graph which depicts the dissolution profile in simulated gastric fluid (SGF) of a 15 mg controlled release oxybutynin formulation as described in Example 6 and as tested according to the procedure described in United States Pharmacopoeia XXIII, Apparatus 2 @ 50 rpm.

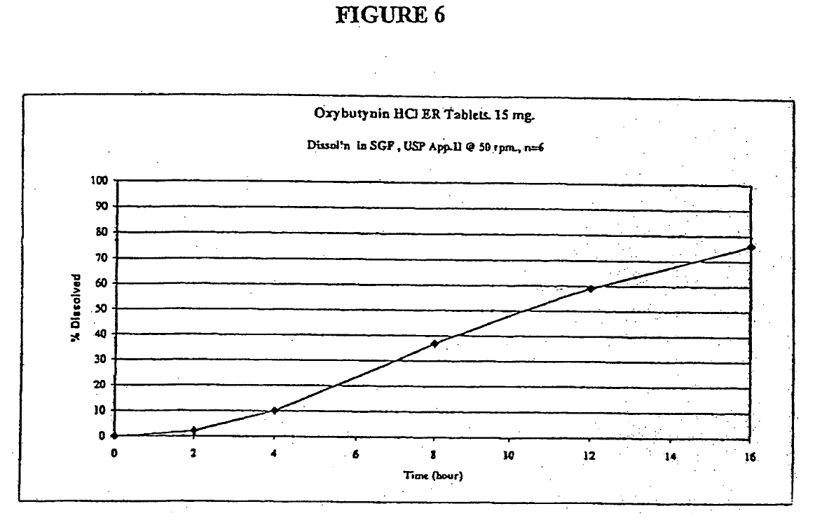

FIG. 7 is a graph which depicts the mean plasma oxybutynin concentration vs. time in the fed state in an N=8 biostudy of Example 3.

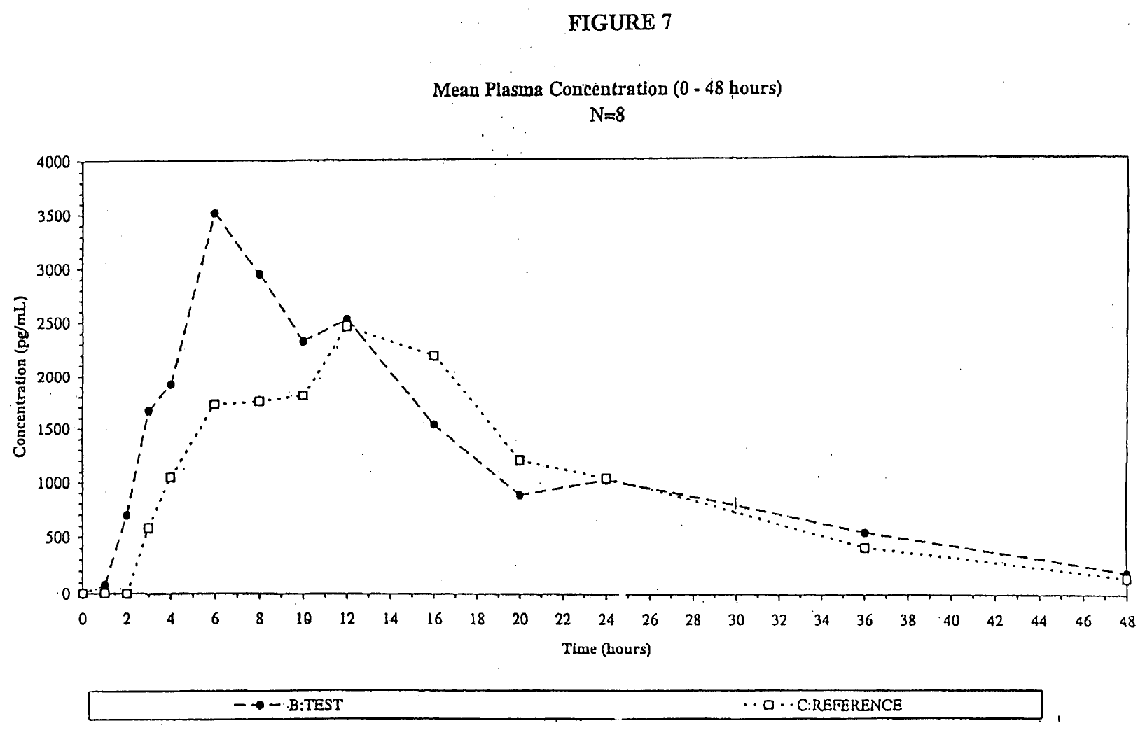

FIG. 8 is a graph which depicts the mean plasma oxybutynin concentration vs. time in the fasted state in an N=8 biostudy of Example 4.

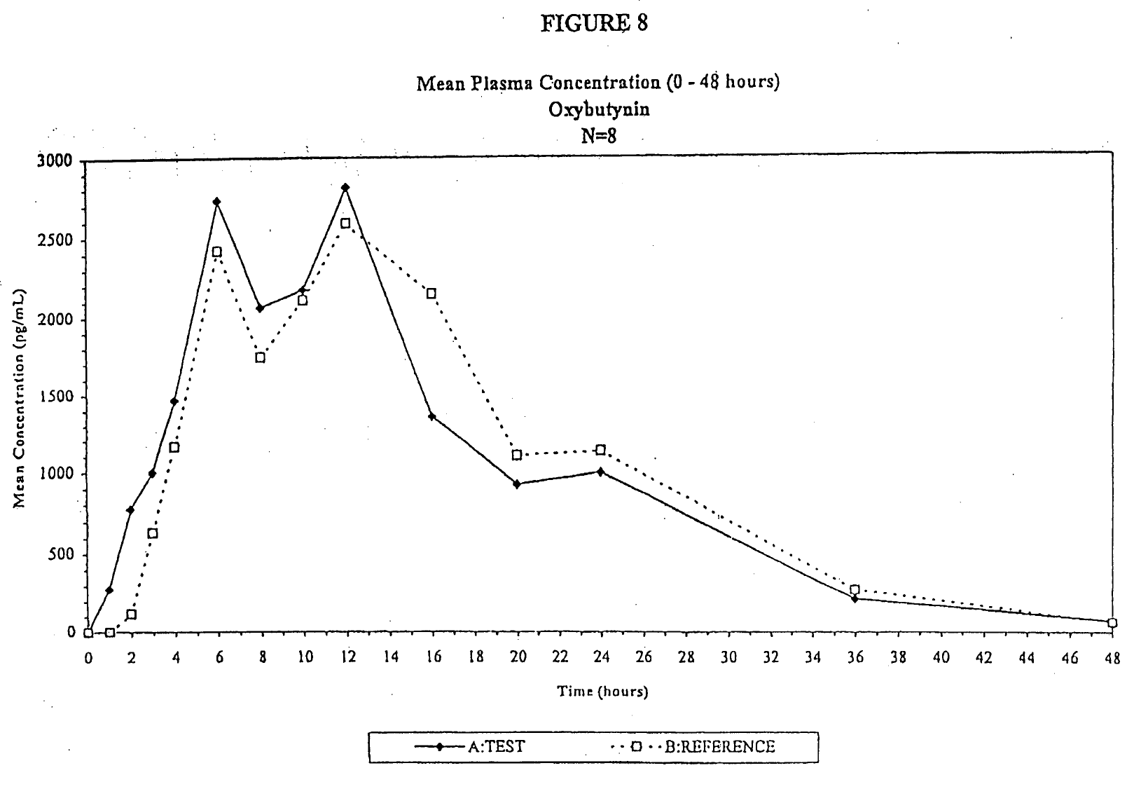

### DETAILED DESCRIPTION OF THE PRESENT INVENTION AND DESCRIPTION OF THE PREFERRED EMBODIMENTS

The present invention should comprise about 1 mg to about 100 mg, preferably 5 mg to about 50 mg of oxybutynin or its pharmaceutically acceptable salts such as the hydrochloride, citrate, succinate, mesylate or fumarate salt. Other pharmaceutically acceptable salts of oxybutynin are disclosed in U.S. Pat. No. 5,674,895 which are incorporated herein by reference. The oxybutynin can be present as the racemate, as the R-enantiomer or as the S-enantiomer. The oxybutynin employed in the present invention may be micronized or unmicronized, or amorphous or crystalline.

The water swellable or hydrogelling material employed in the core is preferably a polymeric material that swells, expands and/or gels when exposed to water. These polymers include polyethylene oxide, methylcellulose, hydroxypropyl cellulose, hydroxypropyl methylcellulose and the like. Other water swellable polymers are disclosed in U.S. Pat. No. 5,912,268 and are incorporated herein by reference. In a preferred embodiment, the water swellable material will be a polyethylene oxide with an average molecular weight between 1 and 7 million, preferable between 3 and 6 million and most preferably about 5 million. Such polyethylene oxide polymers are available from Union Carbide Corporation under the tradename POLYOX®. The water swellable material should be present in the core in an amount less than 50% based upon the total weight of the uncoated core, preferably less than 30% of the uncoated core and most preferably less than 15% of the uncoated core.

Other water swellable polymers that may be used in the present invention include, but are not limited to polyalkylene oxide having a weight average molecular weight of 100,000 to 6,000,000, including but not limited to poly(methylene oxide), poly(butylene oxide); poly(hydroxy alkyl methacrylate) having a molecular weight of from 25,000 to 5,000,000; poly(vinyl)alcohol, having a low acetal residue, which is cross-linked with glyoxal, formaldehyde or glutaraldehyde and having a degree of polymerization of from 200 to 30,000; a mixture of methyl cellulose, cross-linked agar and carboxymethyl cellulose; a hydrogel forming copolymer produced by forming a dispersion of a finely divided copolymer of maleic anhydride with styrene, ethylene, propylene, butylene or isobutylene cross-linked with from 0.001 to 0.5 moles of saturated cross-linking agent per mole of maleic anyhydride in the copolymer; Carbopol® acidic carboxy polymers having a molecular weight of 450,000 to 4,000,000; Cyanamer® polyacrylamides; cross-linked water swellable indenemaleicanhydride polymers; Goodrite® polyacrylic acid having a molecular weight of 80,000 to 200,000; starch graft copolymers; Aqua-Keeps® acrylate polymer polysaccharides composed of condensed glucose units such as diester cross-linked polyglucan and the like. Other polymers which form hydrogels are described in U.S. Pat. No. 3,865,108; U.S. Pat. No. 4,002,173 and U.S. Pat. No. 4,207,893 all of which are incorporated by reference. Mixtures of the aforementioned pharmaceutically acceptable polymers may also be used.

The core of the present invention may also contain an absorption enhancer. The absorption enhancer can be any type of absorption enhancer commonly known in the art such as a fatty acid, a surfactant, a chelating agent, a bile salt or mixtures thereof. Examples of some preferred absorption enhancers are fatty acids such as capric acid, oleic acid and their monoglycerides, surfactants such as sodium lauryl sulfate, sodium taurocholate and polysorbate 80, chelating agents such as citric acid, phytic acid, ethylenediamine tetraacetic acid (EDTA) and ethylene glycol-bis(β-aminoethyl ether)-N,N,N,N-tetraacetic acid (EGTA). The core comprises approximately 0 to about 20% of the absorption enhancer based on the total weight of the core and most preferably about 2% to about 10% of the total weight of the core.

The core may also contain an osmotic apgent. An osmotic agent is any non-toxic pharmaceutically acceptable compound that will increase the osmotic pressure inside the core of the tablet and is preferably a water soluble material. Some of the preferred osmotic agents include the simple sugars and salts such as sodium chloride, potassium chloride, magnesium sulfate, magnesium sulfate, magnesium chloride, sodium sulfate, lithium sulfate, urea, inositol, sucrose, lactose, glucose, sorbitol, fructose, mannitol, dextrose, magnesium succinate, potassium acid phosphate and the like. The preferred osmotic agent for the tablet core is lactose in the range of 40-85% by weight of the core, preferably 50-80% by weight of the core and most preferably 60-75% by weight of the core.

The core may also include other conventional pharmaceutical excipients such as flow agents, e.g. colloidal silicon dioxide, lubricants, e.g. magnesium stearate, diluents, dispersants, surfactants or emulsifiers to optimize the core formulation.

The core of the present invention may be formed by conventional pharmaceutical techniques such as wet granulation, dry granulation or direct compression with the aid of a tablet press.

The homogeneous core is then optionally coated with a protective coating or seal coat. The seal coating may be used at a level in the range of 0-10% by weight of the final dosage form. The seal coating generally comprises a water soluble or rapidly disintegrating polymeric compound such a polyvinyl pyrrolidone (povidone) or polyvinyl alcohols. In an especially preferred embodiment, the polymeric material will be combined with an osmotic agent as described above in the range of 0-10% by weight of the final dosage formulation. While the osmotic agent may be any salts, surfactants or short-chain water soluble polymers, the preferred agent is sodium chloride. The osmotic agent is added to the seal coating system by dissolving or dispersing the polymeric compound and the osmotic agent in a coating solution. This seal coating solution or dispersion is then applied to the homogeneous tablet core by conventional coating techniques such as pan coating or spray coating.

The seal coated or non-seal coated homogeneous core is coated with a semi-permeable membrane, preferably a modified polymeric membrane to form the controlled release tablet of the invention. The semi-permeable membrane is permeable to the passage of an external fluid such as water and biological fluids and is impermeable to the passage of the drug in the core. Polymers that are useful in forming the semi-permeable membrane are cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, and cellulose acetate butyrate. Other suitable polymers are described in U.S. Pat. Nos. 3,845,770; 3,916,899; 4,008,719; 4,036,228 and 4,11210, which are incorporated herein by reference. The most preferred semi-permeable membrane material is cellulose acetate comprising an acetyl content of 39.3 to 40.3%, commercially available from Eastman Fine Chemicals.

In an alternative embodiment, the semi-permeable membrane can be formed from the above-described polymers and a flux enhancing agent. The flux enhancing agent increases the volume of fluid imbibed into the core to enable the dosage form to dispense substantially all of the drug through the passageway and/or the porous membrane. The flux enhancing agent can be a water soluble material or an enteric material. Some examples of the preferred materials that are useful as flux enhancers are sodium chloride, potassium chloride, sucrose, sorbitol, mannitol, polyethylene glycol (PEG), propylene glycol, hydroxypropyl cellulose, hydroxypropyl methylcellulose, poloxamer and mixtures thereof. The flux enhancing agent comprises approximately 0 to about 45% of the total weight of the coating, and most preferably about 5% to about 40% of the total weight of the coating.

In a preferred embodiment of the present invention the flux enhancing agent is a pH sensitive material or enteric polymer. The pH sensitive material or enteric polymer does not begin to dissolve until pH conditions in excess of the stomach region are encountered-by the dosage formulation. Generally, the pH sensitive material or enteric polymer does not begin to dissolve until a pH above 3.0 and preferably above 5.5 is encountered. Materials such as Eudragit L 30 D, Eudragit S 100 or hydroxypropyl methylcellulose phthalate 55 can be used as the flux enhancing agent. Other pH sensitive materials or enteric polymers are described in U.S. Pat. Nos. 5,916,595 and 6,013,281, which are incorporated herein by reference. If a pH sensitive material or enteric polymer is selected as the flux enhancing agent it should comprise 0 to about 35% of the total weight of the semi-permeable membrane, and preferably about 2% to about 30% of the semi-permeable membrane.

In another embodiment of the present invention the semi-permeable membrane comprises a mixture of flux enhancing agents, preferably a pH sensitive material as described above and a water soluble material such as PEG or sodium chloride. The water soluble flux enhancing agent will comprise about 1% to about 20% by weight of the semi-permeable membrane, and preferably about 5% to about 15% by weight of the semi-permeable membrane and the pH sensitive material will comprise about 1% to about 35% of the total weight of the semi-permeable membrane, and preferably about 2% to about 30% of the semi-permeable membrane.

The semi-permeable membrane may also be formed with commonly known excipients such plasticizers. Some commonly known plasticizers include adipate, azelate, enzoate, citrate, stearate, isoebucate, sebacate, triethyl citrate, tri-n-butyl citrate, acetyl tri-n-butyl citrate, citric acid esters and those described in the Encyclopedia of Polymer Science and Technology, Vol. 10 (1969), published by John Wiley & Sons. The preferred plasticizers are triacetin, acetylated monoglyceride, grape seed oil, olive oil, sesame oil, acetyltributylcitrate, acetyltriethylcitrate, glycerin sorbitol, diethyloxalate, diethylmalate, diethylfirmarate, dibutylsuccinate, diethylmalonate, dioctylphthalate, dibutylsebacate, triethylcitrate, tributylcitrate, glyceroltributyrate, and the like. Depending on the particular plasticizer, amounts of from 0 to about 25%, and preferably about 2% to about 15% of the plasticizer can be used based upon the total weight of the coating.

As used herein the term passageway includes an aperture, orifice, bore, hole, weaken area or an erodible element such as a gelatin plug that erodes to form an osmotic passageway for the release of the drug from the dosage form. A detailed description of the passageway can be found in U.S. Pat. Nos. such as 3,845,770; 3,916,899; 4,034,758; 4,077,407; 4,783,337 and 5,071,607.

Generally, the semi-permeable membrane coating around the core will comprise from about 1% to about 10% and preferably about 2% to about 5% based on the total weight of the core and coating. The semi-permeable membrane is applied to the homogeneous tablet core by dissolving or suspending the semi-permeable membrane ingredients in a conventional solvent and applying the solution or suspension onto the homogeneous tablet core by conventional techniques such as pan coating or fluidized bed coating.

Once the semi-permeable membrane is applied the final dosage form may be seal coated, polished or color coated for aesthetic purposes.

The following examples are merely illustrative of the present invention and it should not be considered as limiting the scope of the invention in any way as these examples and other equivalents thereof will become apparent to those versed in the art in light of the present disclosure.

### EXAMPLE 1

A controlled release tablet containing 15 mg of oxybutynin HCl and in accordance with the present invention was prepared as follows:

(a) Homogeneous Tablet Core

3.403 kg of oxybutynin HCl USP, 3.300 kg of polyethylene oxide, NF (POLYOX WSR COAGULANT), 0.165 kg of colloidal silicon dioxide, NF and 24.482 kg of anhydrous lactose, NF are added to a three cubic foot twin shell blender and blended for approximately ten minutes at a speed of 28 rpms. The blended material is then passed through a Comil equipped with a square impeller, an impeller spacing of 175 and operating at a medium speed setting. The blended material is returned to the blender and blended for an additional thirty minutes at a speed of 28 rpms. After the additional blending, 1.32 kg of glyceryl monostearate that has passed through a 30 mesh screen is added to the blender and blended for an additional ten minutes. Finally 0.330 kg of magnesium stearate is passed through a 30 mesh screen and added to the blender. The mixture is then blended for an additional twenty minutes.

The blended mixture is then compressed into tablets using a beta press with a 17/64″ round, standard concave punch that employed a projection in the upper punch. The tablet cores had a target weight of 160 mg and a hardness between 4 to 8 kp. The composition of the resulting tablet cores was:

| INGREDIENT | WEIGHT PERCENT |
|---|---|
| Oxybutynin HCl, USP | 10.312 |
| Polyethylene Oxide*, NF | 10.00 |
| Magnesium Stearate, NF | 1.00 |
| Anhydrous Lactose, NF | 74.188 |
| Colloidal Silicon Dioxide, NF | 0.500 |
| Glyceryl Monostearate, NF | 4.00 |

*POLYOX WSR COAGULANT, with an approximate molecular weight of approximately 5,000,000

(b) Seal Coating (Optional)

Due to the size of available equipment, the tablet cores were divided into three parts or sublots and each sublot comprising about 11 kg of tablet cores was coated with an organic coating solution containing 0.334 kg of povidone K-30, USP, 0.112 kg of sodium chloride, USP, 1.48 kg of acetone, NF and 0.49 kg of isopropyl alcohol, USP. The solution is prepared by adding the povidone to the acetone/isopropyl alcohol solution and homogenizing the solution. Sodium chloride that has been passed through a 100 mesh screen is then added to the solution and homogenized.

Once the coating solution is homogenized, the homogenizer is removed and replaced with a mechanical stirrer and stirred until the coating solution is depleted. The coating solution is sprayed onto the 11 kg sublot of the tablet cores from part (a) using a pan coater (LAB COAT II) employing the following conditions:

| Exhaust air temperature: | 24 ± 5° C. |
|---|---|
| Atomization pressure: | 15-30 psi |
| Air volume: | 150-350 SCFM |
| Pan Speed: | 5-13 rpm |
| Spray Rate: | 7-15 ml/min |
| No. of spray guns: | 2 |
| Nozzle distance | 5″-9″ |

Once the coating solution is consumed, the exhaust air temperature is increased to 30° C. to dry the tablet for about 20 minutes.

(c) Semi-Permeable Membrane Coating

Each of the three seal coaled tablet cores sublots from step (b) are coated with a semi-permeable membrane coating. The semi-permeable membrane coating is prepared by first mixing 5.32 kg of acetone NF and 1.68 kg of isopropyl alcohol (99% pure) USP. To this mixture 0.235 kg of cellulose acetate, NF, 398-10 is added and homogenized for about 5 minutes. 0.117 kg of methacrylic acid copolymer, NTF type B (EUDRAGIT S100) is then added and homogenized. Finally, 0.044 kg of polyethylene glycol 400 NF and 0.044 kg of triacetin, USP is added sequentially and homogenized for approximately two minutes before the next component is added.

The seal coated tablets from step (b) are added to a Glatt fluidized bed coater (GPCG 5) and the tablets are coated with the semi-permeable membrane solution using the following conditions:

| | |
|---|---|
| Product temperature: | 16-28° C. |
| Air velocity: | 600-750 M³/h |
| Spray rate: | 100-200 ml/min |
| Atomization pressure | 2-3 bars |

After the coating solution was consumed the semi-permeable membrane coated tablets were allowed to dry in the fluidized bed for approximately 15 minutes. Each of the three sublots were coated with the semi-permeable membrane using the same conditions.

Once all three of the seal coated sublots were coated with the semi-permeable membrane each sublot was color coated with an aqueous solution of OPADRY PURPLE YS-1-10377. Once all three sublots of the tablet cores from step (a) were color coated, they were recombined and polished using candelilla wax powder.

### EXAMPLE 2

A controlled release tablet containing 15 mg of oxybutynin and having the following formula is prepared as follows:

| I. Core | Percentage of tablet |
|---|---|
| Oxybutynin HCL | 8.44% |
| Magnesium Stearate | 0.97% |
| Anhydrous Lactose | 88.09% |

(a) Core

The oxybutynin and other ingredients comprising the core are blended and pressed into a solid layered core tablet. After blending, the granules are compressed on a rotary press fitted with 17/64″ round standard concave punches (plain lower punch, plain upper punch).

| II. Membrane | Percentage of tablet |
|---|---|
| Cellulose Acetate | 1.35% |
| Eudragit ® S100 | 0.65% |
| Triacetin | 0.25% |
| PEG 400 | 0.25% |

(b) Membrane

The cellulose acetate is dissolved in acetone while stirring with a homogenizer. The Eudragit® S100, polyethylene glycol 400 and triacetin are added to the cellulose acetate solution and stirred until a clear solution is obtained. The clear coating solution is then sprayed onto the seal coated tablets in a fluidized bed coater employing the following conditions: product temperature of 16-22° C.; atomization pressure of approximately three bars; and spray rate of 120-150 ml/min. The sealed core tablet is coated until a theoretical coating level of approximately 3% is obtained.

The resulting tablet is tested in simulated intestinal fluid (pH 6.5 and pH 7.5) according to the procedure described in United States Pharmacopoeia XXIII, Apparatus 2 @ 50 rpm and found to have the following release profile:

| | | % Dissolved (pH 7.5 and |
|---|---|---|
| | % Dissolved (pH 6.5) | Tween 80) |
| Time (Hours) | (n = 3) | (n = 3) |
| 2 | 11 | 16 |
| 4 | 27 | 32 |
| 8 | 45 | 49 |
| 12 | 53 | 57 |
| 16 | 57 | 60 |
| 20 | 59 | 61 |

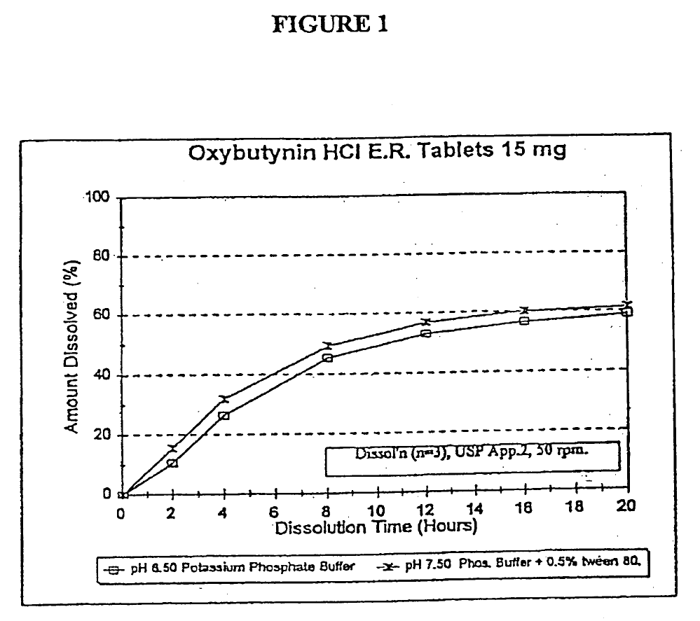

The in-vitro release profile in pH 6.5 and 7.5 of the sustained release product prepared in this Example is shown in FIG. 1.

### EXAMPLE 3

A controlled release tablet containing 15 mg of oxybutynin HCL and having the following formula is prepared as follows:

| I. Core | Percentage of tablet |
|---|---|
| Oxybutynin HCL | 8.16% |
| Polyox WSR Coagulant | 8.71% |
| Magnesium Stearate | 0.87% |
| Anhydrous Lactose | 65.38% |
| Colloidal Silicon Dioxide | 0.44% |
| Glyceryl Monostearate | 3.49% |

(a) Core

The oxybutynin and other ingredients comprising the core are blended and pressed into a solid layered core tablet. After blending, the granules are compressed on a rotary press fitted with 17/64″ round standard concave punches (plain lower punch, upper punch with an approximately 1 mm indentation pin).

(b) Seal Coating (Optional)

The core tablet is seal coated with a coating suspension consisting of povidone, PEG and talc using convention methods in the art. The seal coating will account for 5.56% of the tablet.

| II. Membrane | Percentage of tablet |
|---|---|
| Cellulose Acetate | 2.33% |
| Eudragit ® S100 | 1.16% |
| Triacetin | 0.29% |
| PEG 400 | 0.58% |

(c) Membrane

The cellulose acetate is dissolved in a mixture of acetone and isopropyl alcohol while stirring with a homogenizer. The Eudragit® S100, polyethylene glycol 400 and triacetin are added to the cellulose acetate solution and stirred until a clear solution is obtained. The clear coating solution is then sprayed onto the seal coated tablets in a fluidized bed coater employing the following conditions: product temperature of 16-24. C.; atomization pressure of approximately three bars; and spray rate of 120-150 ml/min. The sealed core tablet is coated until a theoretical coating level of approximately 4.5% is obtained.

| Color Coating | Percentage of Tablet |
|---|---|
| Eudragit ® E100 | 2.69% |
| Titanium Dioxide | 0.23% |
| Candelilla Wax Powder | 0.08% |

(d) Color Coating (Optional)

The membrane coated tablets are color coated with the above color coating formulation and then waxed with candelilla wax powder. The resulting tablet is tested in simulated intestinal fluid (pH 6.5) and simulated gastric fluid (SGF) according to the procedure described in United States Pharmacopoeia XXIII, Apparatus 2 @ 50 rpm and found to have the following release profile:

| | % Dissolved (SGF) | Mean % Dissolved (pH 6.5) |
|---|---|---|
| Time (Hours) | (n = 3) | (n = 3) |
| 2 | 8 | 8 |
| 4 | 22 | 22 |
| 8 | 54 | 48 |
| 12 | 73 | 64 |
| 16 | 88 | 73 |

Table 1 provides mean plasma pharmacokinetic values based on fasting dosing with Example 3 and an equivalent dose of a reference standard (DITROPAN® XL).

TABLE 1
Summary of Statistical Analysis of Example 3 vs. Reference Product
N = 8
Ln-Transformed Data

| | | | | | | | 90% Confidence |
|---|---|---|---|---|---|---|---|
| | Least | | | | | Mean | Level |
| Pk | Squares Means | | Geometric Mean | | | Square | (Lower limit, |
| Variable | Ex. 3 | Ref. | Ex. 3 | Ref. | % ratio | Error | Upper limit |
| Cₘₐₓ | 8.290 | 7.945 | 3985.68 | 2822.23 | 141.22 | 0.09234 | 109.4, 182.3 |
| AUC₀₋ₜ | 10.874 | 10.736 | 52810.94 | 45985.24 | 114.84 | 0.04790 | 95.55, 138.03 |
| AUC_{inf} | 11.019 | 10.871 | 61011.95 | 52643.85 | 115.90 | 0.06750 | 93.17, 144.17 |

### EXAMPLE 4

A controlled release tablet containing 15 mg of oxybutynin HCL and having the following formula is prepared as follows:

| I. Core | Percentage of tablet |
|---|---|
| Oxybutynin HCL | 8.75% |
| Polyox WSR Coagulant | 9.33% |
| Magnesium Stearate | 0.93% |
| Anhydrous Lactose | 70.11% |
| Colloidal Silicon Dioxide | 0.45% |
| Glyceryl Monostearate | 3.73% |
| BHT | 0.02% |

(a) Core

The oxybutynin and other ingredients comprising the core are blended and pressed into a solid layered core tablet. After blending the granules are compressed on a rotary press fitted with 17/64″ round standard concave punches (plain lower punch, upper punch with an approximately 1 mm indentation pin).

(b) Seal Coating (Optional)

The core tablet is seal coated with Povidone K30 and PEG 3350 using convention methods in the art. The Povidone K30 will account for 3.34% of the tablet and PEG 3350 will account for 0.84% of the tablet.

| II. Membrane | Percentage of tablet |
|---|---|
| Cellulose Acetate | 1.35% |
| Eudragit S100 | 0.65% |
| Triacetin | 0.25% |
| PEG 400 | 0.25% |

(c) Membrane

The cellulose acetate is dissolved in acetone while stirring with a homogenizer. The Eudragit S100, polyethylene glycol 400 and triacetin are added to the cellulose acetate solution and stirred until a clear solution is obtained. The clear coating solution is then sprayed onto the seal coated tablets in a fluidized bed coater employing the following conditions: product temperature of 16-22° C.; atomization pressure of approximately three bars; and spray rate of 120-150 ml/min. The sealed core tablet is coated until a theoretical coating level of approximately 3% is obtained. The resulting tablet is tested in simulated intestinal fluid (pH 6.5 and 7.5) according to the procedure described in United States Pharmacopoeia XXIII, Apparatus 2 @ 50 rpm and found to have the following release profile:

| Time (Hours) | % Dissolved (pH 6.5) | Mean % Dissolved (pH 7.5) |
|---|---|---|
| | (n = 3) | (n = 3) |
| 2 | 4 | 5 |
| 4 | 13 | 15 |
| 8 | 33 | 41 |
| 12 | 47 | 61 |
| 16 | 57 | 72 |
| 20 | — | 86 |

Table 2 provides mean plasma pharmacokinetic values based on fasting dosing with Example 4 and an equivalent dose of a reference standard (DITROPAN® XL).

TABLE 2
Summary of Statistical Analysis
N = 8
Ln-Transformed Data

| | Least | | | | | Mean | | 90% | Power |
|---|---|---|---|---|---|---|---|---|---|
| Pk | Squares Means | | Geometric Mean | | | Square | Standard | Confidence | of |
| Variable | Ex. 4 | Ref. | Ex. 4 | Ref. | % ratio | Error | Error | Level | ANOVA |
| Cₘₐₓ | 7.977 | 7.965 | 2.913.18 | 2878.43 | 101.21 | 0.0428 | 0.1034 | 82.8, 124 | 0.4420 |
| AUC₀₋ₜ | 10.457 | 10.645 | 34787 | 41982 | 82.86 | 0.2370 | 0.2434 | 51.6, 133 | 0.1217 |
| AUC_{inf} | 10.565 | 10.805 | 38754 | 49267 | 78.66 | 0.2077 | 0.2279 | 50.5, 122 | 0.1320 |

Geometric means are based on least squares means of log transformed values.

### EXAMPLE5

A controlled release tablet of oxybutynin HCL is prepared in accordance with the procedure of Example 3 with all conditions as described, except that the membrane has the following composition:

| Membrane | Percentage of tablet |
|---|---|
| Cellulose Acetate | 2.10% |
| Eudragit ® S100 | 1.60% |
| Triacetin | 0.29% |
| PEG 400 | 0.58% |

The resulting tablet of Example 5 is tested in simulated gastric fluid (SGF) and simulated intestinal fluid (pH 6.5) according to the procedure described in United States Pharmacopoeia XXIII apparatus 2 @ 50 rpm and found to have the following release profile:

| Time (Hours) | % Dissolved (SGF) | % Dissolved (pH 6.5) |
|---|---|---|
| 2 | 5 | 7 |
| 4 | 18 | 19 |
| 8 | 47 | 50 |
| 12 | 68 | 67 |
| 16 | 87 | 76 |

### EXAMPLE 6

A controlled release tablet of oxybutynin HCL is prepared in accordance with the procedure of Example 5, except that the seal coating and membrane coating compositions are as follows:

| Seal Coating Composition | Percentage of seal coating |
|---|---|
| Povidone K30 | 75.0% |
| Talc | 25.0% |

| Membrane | Percentage membrane coating |
|---|---|
| Cellulose Acetate | 60.00% |
| Eudragit ® S100 | 20.00% |
| Triacetin | 6.68% |
| PEG 400 | 13.32% |

6% of the seal coat and 8% of the membrane coating are theoretically coated onto the core tablets, respectively. The resulting tablet is tested in simulated gastric fluid (SGF) according to the procedure described in the USP apparatus 2 @ 50 rpm and found to have the following release profile.

| Time (Hours) | % dissolved (SGF) |
|---|---|
| 2 | 2 |
| 4 | 10 |
| 8 | 37 |
| 12 | 59 |
| 16 | 76 |

### EXAMPLE 7

The procedures of Examples 3 to 5 are followed except that LustreClear™ (a microcrystalline cellulose/carrageenan based coating system) is used for seal coating.

### EXAMPLE 8

The procedure of Examples 3 to 5 is followed, except that Kollicoat® IR (a polyvinyl alcohol-polyethylene glycol graft copolymer) is used for seal coating.

### EXAMPLE 9

The procedure of Example 2 is followed, except that the color coating composition is as follows:

| Membrane | % membrane coating |
|---|---|
| Kollicoat ® IR | 89.53% |
| Titanium Dioxide | 7.83% |
| Lake Blend Purple | 2.64% |

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

1. A controlled release pharmaceutical tablet comprising:
(a) a homogeneous core comprising:
(i) an effective amount of oxybutynin or a pharmaceutically acceptable salt;
(ii) less than 50% by weight of the core of a water swellable or water gelling material;
(b) optionally a seal coat surrounding the core;
(c) a semi-permeable membrane that surrounds the core; and
(d) at least one passageway in the semi-permeable membrane.

2. The controlled release pharmaceutical tablet as defined in claim 1 wherein the amount of water swellable or water gelling material in the core is less than 30% by weight of the core.

3. The controlled release pharmaceutical tablet as defined in claim 1 wherein the amount of water swellable or water gelling material in the core is less than 15% by weight of the core.

4. The controlled release pharmaceutical tablet as defined in claim 1 wherein the water swellable material is selected from the group consisting of polyethylene oxide, methylcellulose, hydroxypropyl cellulose and hydroxypropyl methylcellulose.

5. The controlled release pharmaceutical tablet as defined in claim 1 wherein the water swellable material is polyethylene oxide with an approximate molecular weight between 1 and 7 million

6. The controlled release pharmaceutical tablet as defined in claim 5 wherein the polyethylene oxide has an approximate molecular weight between 3 and 6 million.

7. The controlled release pharmaceutical tablet as defined in claim 6 wherein the polyethylene oxide has an approximate molecular weight of about 5 million.

8. The controlled release pharmaceutical tablet as defined in claim 1 wherein the core further comprises an absorption enhancer.

9. The controlled release pharmaceutical tablet as defined in claim 8 wherein the absorption enhancer comprises about 2% to about 10% of the total weight of the core.

10. The controlled release pharmaceutical tablet as defined in claim 8 wherein the absorption enhancer is selected from the group consisting of a fatty acid, a surfactant, a chelating agent, a bile salt or mixtures thereof.

11. The controlled release pharmaceutical tablet as defined in claim 1 wherein the core further comprises an osmotic agent.

12. The controlled release pharmaceutical tablet as defined in claim 11 wherein the osmotic agent comprises about 40% to about 85% of the total weight of the core.

13. The controlled release pharmaceutical tablet as defined in claim 12 wherein the osmotic agent comprises about 50% to about 80% of the total weight of the core.

14. The controlled release pharmaceutical tablet as defined in claim 13 wherein the osmotic agent comprises about 60% to about 75% of the total weight of the core.

15. The controlled release pharmaceutical tablet as defined in claim 11 wherein the osmotic agent is selected from the group consisting of sodium chloride, potassium chloride, magnesium sulfate, magnesium sulfate, magnesium chloride, sodium sulfate, lithium sulfate, urea, inositol, sucrose, lactose, glucose, sorbitol, fructose, mannitol, dextrose, magnesium succinate, potassium acid phosphate and mixtures of the foregoing.

16. The controlled release pharmaceutical tablet as defined in claim 1 wherein the core further comprises conventional pharmaceutical excipients selected from the group consisting of flow agents, lubricants, diluents or mixtures of the foregoing.

17. The controlled release pharmaceutical tablet as defined in claim 1 wherein the seal coating comprises 0-10% by weight of the total weight of the controlled release tablet.

18. The controlled release pharmaceutical tablet as defined in claim 17 wherein the seal coating comprises a water soluble or rapidly disintegrating polymeric compound and an osmotic agent.

19. The controlled release pharmaceutical tablet as defined in claim 1 wherein the semi-permeable membrane comprises:
(i) a polymer that is permeable to the passage of water and biological fluids and is impermeable to the passage of the oxybutynin in the core; and
(ii) a flux enhancing agent.

20. The controlled release pharmaceutical tablet as defined in claim 19 wherein the flux enhancing agent is selected from the group consisting of water soluble materials or enteric materials.

21. The controlled release pharmaceutical tablet as defined in claim 20 wherein the water soluble flux enhancing agent is selected from the group consisting of sodium chloride, potassium chloride, sucrose, sorbitol, mannitol, polyethylene glycol, propylene glycol, hydroxypropyl cellulose, hydroxypropyl methycellulose and mixtures thereof.

22. The controlled release pharmaceutical tablet as defined in claim 19 wherein the flux enhancing agent is a combination of water soluble materials and enteric materials.

23. The controlled release pharmaceutical tablet as defined in claim 22 wherein the water soluble materials comprise about 1% to about 20% of the total weight of the semi-permeable membrane and the enteric materials comprise about 1% to about 35% of the total weight of the semi-permeable membrane.

24. The controlled release pharmaceutical tablet as defined in claim 23 wherein the water soluble materials comprise about 5% to about 15% of the total weight of the semi-permeable membrane and the enteric materials comprise about 2% to about 30% of the total weight of the semi-permeable membrane.

25. The controlled release pharmaceutical tablet as defined in claim 19 wherein the semi-permeable membrane further comprises a plasticizer.

26. A controlled release pharmaceutical tablet consisting essentially of:
(a) a homogeneous core consisting essentially of:
(i) an effective amount of oxybutynin or a pharmaceutically acceptable salt;
(ii) less than 50% by weight based upon the total weight of the core of a water swellable or water gelling polymer;
(iii) 0-20% based on the total weight of the core of an absorption enhancer;
(iv) 40-85% based upon the total weight of the core of an osmotic agent;
(v) optionally a flow aid;
(vi) optionally a lubricant;
(b) optionally a seal coat surrounding the core;
(c) a semi-permeable membrane that surrounds the core consisting essentially of
(i) a polymer that is permeable to the passage of water and biological fluids and is impermeable to the passage of the oxybutynin in the core;
(ii) 5-40% based upon the total weight of the semi-permeable membrane of a flux enhancing agent; and
(iii) optionally a plasticizer; and
(d) at least one passageway in the semi-permeable membrane.

27. The controlled release pharmaceutical tablet as defined in claim 26 wherein the amount of water swellable or gelling polymer in the core is less than 30% by weight of the core.

28. The controlled release pharmaceutical tablet as defined in claim 26 wherein the amount of water swellable or gelling polymer in the core is less than 15% by weight of the core.

29. The controlled release pharmaceutical tablet as defined in claim 28 wherein the water swellable polymer is a polyethylene oxide having an approximate molecular weight between 1 and 7 million.

30. The controlled release pharmaceutical tablet as defined in claim 28 wherein the water swellable polymer is a polyethylene oxide having an approximate molecular weight between 3 and 6 million.

31. The controlled release pharmaceutical tablet as defined in claim 26 wherein the flux enhancing agent is a combination of water soluble materials and enteric materials.

32. The controlled release pharmaceutical tablet as defined in claim 31 wherein the water soluble materials comprise about 1% to about 20% of the total weight of the semi-permeable membrane and the enteric materials comprise about 1% to about 35% of the total weight of the semi-permeable membrane.

33. The controlled release pharmaceutical tablet as defined in claim 32 wherein the water soluble materials comprise about 5% to about 15% of the total weight of the semi-permeable membrane and the enteric materials comprise about 2% to about 30% of the total weight of the semi-permeable membrane.

34. A controlled release pharmaceutical tablet consisting essentially of:
(a) a homogeneous core consisting essentially of:
(i) an effective amount of oxybutynin or a pharmaceutically acceptable salt;
(ii) less than 30% by weight based upon the total weight of the core of a water swellable or water gelling polymer;
(iii) 2-10% based on the total weight of the core of an absorption enhancer;
(iv) 50-80% based upon the total weight of the core of an osmotic agent;
(v) optionally a flow aid;
(vi) optionally a lubricant;
(b) optionally a seal coat surrounding the core;
(c) a semi-permeable membrane that surrounds the core consisting essentially of
(i) a polymer that is permeable to the passage of water and biological fluids and is impermeable to the passage of the oxybutynin in the core;
(ii) 5-40% based upon the total weight of the semi-permeable membrane of a flux enhancing agent; and
(iii) optionally a plasticizer; and
(d) at least one passageway in the semi-permeable membrane.

35. The controlled release pharmaceutical tablet as defined in claim 34 wherein the amount of water swellable or gelling polymer in the core is less than 15% by weight of the core.

36. The controlled release pharmaceutical tablet as defined in claim 34 wherein the water swellable polymer is a polyethylene oxide having an approximate molecular weight between 3 and 6 million.

37. The controlled release pharmaceutical tablet as defined in claim 34 wherein the flux enhancing agent is a combination of water soluble materials and enteric materials.

38. The controlled release pharmaceutical tablet as defined in claim 37 wherein the water soluble materials comprise about 1% to about 20% of the total weight of the semi-permeable membrane and the enteric materials comprise about 1% to about 35% of the total weight of the semi-permeable membrane.

39. The controlled release pharmaceutical tablet as defined in claim 38 wherein the water soluble materials comprise about 5% to about 15% of the total weight of the semi-permeable membrane and the enteric materials comprise about 2% to about 30% of the total weight of the semi-permeable membrane.
